(19) United States
(12) Patent Application Publication
(10) Pub. No.: US 2015/0059770 A1
(43) Pub. Date: Mar. 5, 2015

(54) TOOLS, DEVICES AND METHODS FOR INTERMAXILLARY FIXATION

(71) Applicant: Mercy Medical Research Institute, Springfield, MO (US)

(72) Inventors: Bharat Shah, Springfield, MO (US); Keela Davis, Springfield, MO (US); Anna Kelley, Walnut Grove, MO (US); Rahul Eapen, Monett, MO (US)

(21) Appl. No.: 14/473,726

(22) Filed: Aug. 29, 2014

Related U.S. Application Data
(60) Provisional application No. 61/871,456, filed on Aug. 29, 2013.

(51) Int. Cl. A61B 17/66; A61C 5/14

(52) U.S. Cl. CPC A61B 17/663; A61C 5/14
USPC 128/861; 606/54

(57) ABSTRACT

Novel tools, accessories and methods for fixing a patient's upper and lower jaws together with flexible straps are disclosed. The tools are intended to either allow placement of a flexible strap to bind items to the teeth, or for using a flexible strap to fasten the upper and lower jaw into place, among other purposes. These accessories include a washer with a ratchet head for mounting on a bone screw, a bone screw with integrated ratchet head, dental blocks for use with the bone screws and washers, a flexible strap provided with a dissection tip for forcing the strap through tissue, and combinations thereof, and tools for inserting flexible straps through the gums, among other tools and accessories.

### CROSS-REFERENCE TO RELATED APPLICATIONS

This application claims the benefit of U.S. Patent Application Ser. No. 61/871,456 filed Aug. 29, 2013, the disclosure of which is incorporated herein by reference.

### BACKGROUND

This invention relates generally to tools, devices and improved methods for providing intermaxillary fixation. The benefits of intermaxillary fixation, rigidly connecting the upper jaw to the lower jaw, are well known to promote healing of jaw fractures. Intermaxillary fixation is a common treatment to stabilize jaws for oral, plastic, maxillofacial, ENT and trauma surgeries. Further, intermaxillary fixation has been disclosed as a preferred stabilization method for the short term treatment of facial fractures in non-surgical settings, such as on the battlefield by military corpsmen.

The general process of fixing the upper and lower jaw together for medical purposes has been in place for decades. Early efforts for intermaxillary fixation were rudimentary, generally ineffective, often exacerbated patient discomfort, and often failed to achieve desired results. An early process for intermaxillary fixation included the steps of boring holes through the patient's upper and lower gums, passing a wire through the holes and then twisting the wire to join the upper jaw to the lower jaw. That process required boring several holes through the patient's gums. Later efforts have included a variety of apparatuses and processes that include drilling holes, placing bolts, screws or anchors in to the patient's gums, jaw bones and palate. Although such processes increase the possibility of patient discomfort, infection and long-term bone and tissue damage, they are sometimes necessary to effectively fix the patient's jaws together.

These methods have improved over the years including the device disclosed and described in U.S. Pat. No. 8,414,581 to Shah et al. Contemporary treatment methods tend to use non-invasive procedures when possible. The development of the Shah device was a significant advancement in the art in that it uses arch bars that fit adjacent to the outer surface of the patient's upper and lower teeth and are then fastened in place using a plurality of pliable zip or cable ties that connect the arch bars to individual teeth. Interarch receptacles provided on both the upper and lower arch bars can then be fastened together using reverse zip ties or double-ended flexible straps (described herein) thus holding the upper and lower jaw together. In many situations, this device eliminates the need for a physician to drill in to the patient's gum, palate or jaw to fix the upper and lower jaws together. The use of arch bars and zip tie connectors allows for easy and quick installation, removal and repair of the device.

However, the Shah device works best when patients have substantially all of their teeth. When patients present with substantial trauma to their jaw and teeth or have multiple teeth removed or missing, then the interarch bar may not have enough support on the remaining teeth to be an effective treatment. Likewise, for patients that have dentures or no teeth whatsoever, the Shah device may be ineffective as disclosed and described in the previously referenced Shah patent.

Accordingly, it is beneficial to provide a treatment that utilizes, to the extent possible, the noninvasive treatment disclosed in the previously referenced Shah patent, along with tools, devices and procedures that facilitate intermaxillary fixation when a patient has less than all of their teeth, dentures or no teeth at all. While the elimination of bone screws, drilling, boring and wire placement is a desirable goal, some of the historic techniques can be used in conjunction with more modern techniques and devices to substantially lessen the invasiveness of the intermaxillary fixation process.

### SUMMARY OF THE INVENTION

The instant invention is intended to overcome certain limitations that are present in the noninvasive intermaxillary fixation devices, such as the Shah device. More specifically, the invention is intended to meet the needs of patients who do not have enough teeth for the intermaxillary fixation arch bar system to work effectively and in instances where the patient presents with dentures, partial teeth or no teeth at all. The inventive tools, devices and methods disclosed herein utilize the best features of arch bar fixation devices, such as that disclosed by Shah, with some traditional methods that can be effective in those circumstances described.

For example, a patient presenting with most of their teeth, but lacking teeth in one specific area of their mouth, a relatively common occurrence from trauma sustained in vehicle collisions or resulting from a sports injury, interarch bars can be used in those areas of the mouth where most of the teeth are present. However, in the area where teeth are not present, there would be insufficient stability to support an arch bar connected with an interarch receptacle and a strap with serrations.

When described herein the female end of a zip or cable tie may be referred to as ratchet head, or as an interarch receptacle. Both terms refer to the case having a port through it for receiving a flexible strap. A flexible retention member or pawl is disposed inside the port of the ratchet head or interarch receptacle, as is commonly found with the female end of a zip or cable tie. The male end of a zip or cable tie may be referred to as a flexible strap. The flexible straps are provided with ratchet teeth or serrations. Some embodiments of the flexible strap are attached at one end to a ratchet head forming the common cable or zip tie. Some embodiments of the flexible straps are attached at one end to an inventive washer described in relation to the figures. Some embodiments of the flexible straps have two male ends, each with a section of ratchet teeth oriented in opposing directions, which may be referred to as a double-ended flexible strap. Each end of the double-ended flexible strap may be inserted into opposing interarch receptacles to fix the patient's jaws together, as is described in more detail in relation to the figures. The various embodiments of the flexible straps are inserted through the port on the ratchet head, thereby engaging the ratchet teeth on the flexible strap with the pawl and allowing insertion of the flexible strap but not its removal from the port. In some embodiments the flexible straps are flexible along their length, in one or both axes perpendicular to the longitudinal axis of the strap, so that they may be bent, curved or twisted in simple or compound curves as necessary. In some embodiments the flexible straps have limited extensibility or compressibility parallel to their longitudinal axis.

In another embodiment of the invention, a unique hanger assembly provides a ratchet head, similar to those found on the Shah arch bar assembly, which can be connected to a screw fastened in to the patient's jaw bone. As shown in FIG. 1, a washer, or preferably, a slotted washer incorporated into a hanger can be placed onto the bone screw and secured in place by the screw head as the screw is inserted and tightened. The preferred washer includes an integrally formed ratchet head or interarch receptacle. In some embodiments, the screw would be positioned opposite an arch bar attached to existing teeth on the opposing jaw, so that the ratchet head would be presented opposite to an interarch receptacle mounted on the arch bar fastened to the existing teeth. The ratchet head may then be secured to the interarch receptacle using a double-ended flexible strap.

For example, if the patient presented with no teeth on the left upper jaw but with teeth on the lower left jaw, an arch bar, similar to the Shah device, can be fastened to the lower teeth. At least one bone screw would be mounted into the upper left jaw bone of the patient opposite each arch bar receptacle provided on the arch bar fastened to the left lower teeth. An inventive washer assembly, an embodiment of which is depicted in FIGS. 1A, 1B, and 1C, may either be placed on the screw and simultaneously mounted with the bone screw, or slipped over the bone screw in the case of a slotted washer, and then secured in place by tightening the bone screw. The ratchet head incorporated into the washer assembly may be oriented so that a double-ended flexible strap can be inserted into both the ratchet head attached to the upper jaw, and to the interarch receptacle attached to the lower jaw so that when the patient's jaws are closed together the double-ended flexible strap holds the opposing arch bar receptacles and associated arch bar (on the lower teeth) in place.

In situations where multiple teeth are missing from both the upper and lower jaw of the patient, it may be necessary to use multiple bone screws on both the upper and lower jaw to present enough ratchet heads or interarch receptacles so that the patient's jaws are maintained in the preferred rigid closed position by the flexible straps.

In another embodiment of an intermaxillary fixation accessory, the innovative washer for fastening to a bone screw may also be attached to a flexible strap, an embodiment of which is shown in FIG. 1C. When a bone screw is positioned opposite an interarch receptacle or a washer hanger assembly attached to the other jaw, the male end of the flexible strap may be inserted into the opposing receptacle to secure the two bone screws and thus the two jaws. A physician may alternate using the washer hanger assembly with either a washer with attached flexible strap or another washer hanger assembly and a double-ended flexible strap, as is most suitable for the application and circumstances.

In some embodiments the bone screw must be inserted through the washer prior to placing the bone screw in the jaw. In other embodiments, the washer component is a slotted washer that has one end of its slot large enough to pass over the head of the bone screw and the other end of the slot small enough that it will not pass over the head of the bone screw. This allows the bone screw to be placed in the jaw and then the washer passed over the screw head and then manipulated so that the narrow portion of the slot underlies the head of the screw such that it cannot be removed without loosening or removing the bone screw. This also allows the washer assembly to be removed from the bone screw without fully removing the screw, in instances where the fastening assembly must be adjusted or replaced. Substantial care must be taken when applying washers with bone screws to not unduly compress gum tissue as doing so can cause permanent damage to the patient's gums.

Another novel tool for intermaxillary fixation is a flexible strap-compatible bone screw. As shown in FIGS. 2A, 2B, 2C, and 2D, the bone screw includes a threaded shaft and an elongated driving head for engagement with a driving tool as is common in the practice of seating bone screws. The driving head includes a ratchet head. The ratchet head may be integrally formed with the head of the bone screw or inserted into a cavity in the head of the bone screw. In some embodiments the ratchet head is inserted into and frictionally retained in a cavity or opening extending laterally through the elongated driving head of the screw. In some embodiments, the ratchet head may include a small ledge or other retention member along one edge of the case thereof to further support and retain the ratchet head in place. In other embodiments, as shown in FIG. 2D, the ratchet head may be formed with at least one lip or protrusion that engages a portion of the head of the bone screw to prevent the ratchet head from passing through the cavity in the head of the bone screw.

The inventive screw is installed in a traditional manner by driving the threaded portion of the shaft in to the bone of the patient. In an embodiment with the ratchet head incorporated into the head of the bone screw, the openings of the ratchet head are aligned as needed for clamping the jaws together, and are often oriented in an up and down orientation, with respect to the jawline of the patient. A flexible strap is inserted into the ratchet head. When the bone screw is mounted in the lower jaw, the orientation of the ratchet head and flexible strap is reversed.

In another embodiment the ratchet head is not integrally formed with the bone screw and must be inserted into the cavity in the head of the bone screw. When this embodiment of the bone screw is installed in the jaw of a patient, the openings of the cavity and the optional retaining ledge are oriented generally toward the opposite jaw. The ratchet head is then installed into the cavity. Any protrusions on the ratchet head are disposed away from the opposing jaw so that the pulling force exerted by the flexible strap will pull the ratchet head farther into the cavity in the bone screw. In some circumstances, bone screws are presented in both the upper and lower jaw and are substantially aligned. A double-ended flexible strap may be provided and inserted into the ratchet heads on both bone screws. Thus, as the double-ended flexible strap is pulled upward through the ratchet head in the top bone screw and downward through the ratchet head in the lower bone screw, the patient's jaws are forced together.

For those instances where a patient presents with upper, lower or both upper and lower dentures, the full interarch bar fixation assembly may not be effective because the dentures do not afford the stability necessary for good intermaxillary fixation. It is desirable, however, to fix the jaw in place with the dentures inserted so that as jaw bones heal they are healed in the configuration, orientation and spacing necessary to accommodate post-procedure denture usage that is appropriate and patient friendly. Fixing the jaws together without the dentures in place would likely result in improper post-procedure denture fit because the bone alignment would be different from the bone alignment at the time the dentures are originally fitted.

Some current methods for intermaxillary fixation when a patient has dentures involve an elaborate process of wiring the dentures in place. For the lower dentures, a hole is generally made between the patient's gum and cheek tissue oriented downward and passing along the patient's jaw, exiting under the chin. A second hole is then bored upward through the floor of the patient's mouth adjacent the inner edge of the jaw and gums. This bore is generally made with a pointed or sharp instrument such as an awl or trochar. The dentures are then placed over the patient's gums and a wire is inserted through the hole between the patient's cheek and gum, passed downward below the patient's chin and then back upward and through the bore that is interior the patient's jaw bone. The wire is passed over the dentures and twisted or otherwise secured to itself so that the dentures are pulled downward on to the patient's gum and retained rigidly in place. A plurality of these circum-mandibular wires may be necessary to rigidly fix the lower dentures in place. In some cases this requires two sets of holes on either side of the patient's mouth, and in practice as many as four to six sets of holes may be used.

For dentures on the upper jaw, multiple bone screws through the patient's hard palate can be used to secure the dentures in place. In the alternative, holes can be bored in the upper gum to allow wires to pass through these holes in the piriform aperture adjacent to the nose, and these wires are then tied around the circum-mandibular wires (wires around the lower jaw). Another alternative is to fix screws into the patient's upper jaw and then fastening wires to the screws which are then secured to bone screws in the lower jaw bone or, in some instances, to additional wires passed through holes drilled in the gum line (circummandibular wires).

The use of wires to tie the dentures in place is not desirable because of the difficulty in positioning the wires, the propensity of the wires to cause sores within the patient's mouth and for the ends of the wires to gouge patient's jaws, cheek, tongue and the like. Moreover, as is well known, the use of wires in a patient's mouth often results in injuries to the physician, orthodontist and other medical staff during placement, adjustment and removal. One of the inventive devices that overcomes many of these limitations and drawbacks is an improved zip tie assembly that can be substituted for the wires in the process described above. In one instance, shown in FIG. 4, a flexible strap is presented that has a dissection tip at one end of the strap. This dissection tip allows the strap to be pushed through the tissues. The dissection tip is preferably formed from extremely rigid and sharp plastic, but can have an integrated metal cutting end formed from materials commonly found in scalpels, needles and the like. In some embodiments the dissection tip is sharp enough to easily pass through tissue, but not so sharp that it severs nerves and vessels. In some embodiments the dissection tip is molded to the flexible strap but is easily removed by cutting apart the plastic portion of the flexible strap adjacent the tip.

In practice, the dissection tip of the flexible strap is used in a similar manner to an awl or trochar for creating tissue access for wire placement. The flexible strap is pushed, passed or guided through the tissue adjacent the gum and then passed through a small incision made below the patient's chin and then back up through the tissue thereby encircling the lower jaw and any associated dentures or dental blocks. Likewise, for application on an upper jaw of a patient, bores through the patient's gum, such as commonly used for that application are formed. A flexible strap can then be positioned through the bore around the jaw and fastened to a ratchet head on the other end of the flexible strap to hold the upper dentures in place. Multiple bores with multiple fasteners may be necessary to firmly secure dentures to the patient's upper gums. Once the dentures are fastened in place, the upper and lower jaws can be fixed together by traditional means, or by fastening interarch bars to the upper and lower teeth, whether natural or dentures, and then using an interarch bar attachment assembly such as that disclosed in the Shah patent. Alternatively, the innovative washer attachment system described herein may be utilized for intermaxillary fixation once the dentures are secured in place.

In those instances when a patient presents with no teeth whatsoever and dentures are not provided or otherwise available, another embodiment of the invention may be used to securely fix the patient's jaws. As described above, it is not desirable to fix the patient's upper and lower jaws together for bone healing when there are no teeth or dentures present. Doing so results in improper jaw bone alignment during the healing process and may make it difficult, if not impossible, for subsequent use of dental implants or dentures. In other embodiments of the invention, as shown in FIGS. 3A, 3B, 6A, and 6B, a unique set of dental blocks has been designed and developed to support the jaws and provide the proper spacing. One dental block is suitable for placement for the upper teeth, shown in FIG. 3A, and the other block is designed for the lower teeth as shown in FIG. 3B. The blocks are comprised of rigid or semi-rigid plastic portion that is sized to simulate the patient's teeth and a soft, formable or malleable portion, sometimes formed from plastic, that engages the patient's gum when the block is in place. When in place, the dental block holds the upper and lower jaw apart a sufficient distance such that when broken jaw bones are healed, suitable space has been provided between the jaw bones for proper placement of dental implants or dentures. In some embodiments, a single dental block may contact both upper and lower gums for a patient with no teeth.

As shown in FIGS. 3C, 3D, 6A, and 6B, each dental block is provided with multiple receptacles for receiving the male portion of a zip tie. The dental block is held in place using methods described above for securing dentures in place. The upper dental block may include screw holes for fastening the block in place with screws placed in the patient's palate. Once the dental blocks are fixed in place, double-ended flexible straps can be used in the receptacles on the dental blocks to fasten the upper and lower jaws together.

Another inventive tool is a unique trochar or awl that is provided with a unique retention member at or substantially near the blade or dissection tip of the awl. The retention member is configured to engage and frictionally retain a knob provided on another embodiment of the flexible strap which may be used as a circum-mandibular strap. In some embodiments, a slot may be presented in the blade of the awl to engage a pin attached to the circum-mandibular strap being placed through tissue contemporaneously with advancement of the awl through the tissue. The awl is used in a manner similar to a trochar or awl would be used to form a path around the patient's jaw for securement of dentures or a dental block as described above. Once the circum-mandibular cable has been placed around the patient's jaw as described, the cable is disconnected from the awl by simply disengaging the pin on the strap from the retention member.

### BRIEF DESCRIPTION OF DRAWINGS

FIG. 1A is a perspective view of an embodiment of an intermaxillary fixation accessory.

FIG. 1B is an exploded perspective view of an embodiment of the Intermaxillary fixation accessory of FIG. 1A.

FIG. 1C is a perspective view of an embodiment of a pair of opposed intermaxillary fixation accessories.

FIG. 2A is a perspective view of an embodiment of a bone screw for intermaxillary fixation with a ratchet head.

FIG. 2B is a top perspective view of the embodiment of the bone screw of FIG. 2A.

FIG. 2C is a side perspective view of the embodiment of the bone screw of FIG. 2A.

FIG. 2D is a partial exploded view of an embodiment of the bone screw and ratchet head of FIG. 2A.

FIG. 3A is a perspective view of an embodiment of an upper dental block for intermaxillary fixation.

FIG. 3B is a perspective view of an embodiment of a lower dental block for intermaxillary fixation.

FIG. 3C is a perspective view of an embodiment of upper and lower dental blocks for intermaxillary fixation.

FIG. 3D is another perspective view of an embodiment of the upper and lower dental blocks for intermaxillary fixation.

FIG. 3E is a perspective view of an embodiment of a ratchet head with clip component.

FIG. 3F is a bottom perspective view of an embodiment upper dental block shown in FIG. 3A.

FIG. 3G is a bottom perspective view of an embodiment of the lower dental block shown in FIG. 3B.

FIG. 3H is a perspective view of an embodiment of a dental block.

FIG. 3I is a perspective view of the embodiment of a dental block of FIG. 3H.

FIG. 4 is a partial perspective view of an embodiment of a circum-mandibular strap with ratchet head and serrations and a blade for piercing tissue in certain procedures for intermaxillary fixation.

FIG. 5A is a perspective view of an embodiment of a tool for piercing tissue and inserting a circum-mandibular strap.

FIG. 5B is a perspective view of an embodiment of the tool of FIG. 5A and a modified circum-mandibular strap attached to the tool.

FIG. 5C is a detailed perspective view of a portion of the embodiment of the tool and strap shown in FIG. 5B.

FIG. 6A is a top perspective view of an additional embodiment of a dental block.

FIG. 6B is a bottom perspective view of an additional embodiment of a dental block.

### DETAILED DESCRIPTION OF THE INVENTION

Referring now generally to the drawings, the instant invention relates to devices to improve intermaxillary fixation for patients who do not have all, or any, of their teeth. The inventive devices and methods disclosed herein may be utilized separately or in conjunction with arch bar fixation devices described in the Shah patent to provide intermaxillary fixation for patients missing some or all of their teeth.

In various embodiments of the devices disclosed herein, various ratchet components and straps with ratchet teeth are described. In the depicted embodiments, the straps are provided on one surface with a gear rack section provided with a plurality of ratchet teeth or serrations. A first or male end of the strap may have a narrowing or pointed end or a blunt end. The strap itself may be a flat tape, have a circular cross-section, or other similar shapes. The strap is preferably formed from a flexible but strong material. In some embodiments, a second end of the strap is provided with a ratchet head with a port through the head. Inside the port, a pawl is provided to engage ratchet teeth disposed on the strap. When the first end of the strap is inserted through the port in the ratchet head, the ratchet teeth engage the pawl to allow the strap to be pulled through the port but not to be retracted from the port.

For clarity, the second end with the ratchet head, sometimes referred to as the female end, of a zip tie or strap may be referred to herein as an interarch receptacle and the first or male end may be referred to as a flexible strap provided with ratchet serrations or teeth. It should be understood that the interarch receptacle includes a port and a flexible retention member, or pawl, as is commonly found with the ratchet head, or female end, of a cable or zip tie.

Some embodiments of the flexible straps are attached at one end to an inventive washer described in relation to the figures. Some embodiments of the flexible straps have two male ends, each with a section of ratchet teeth oriented in opposing directions, which may be referred to as a double-ended flexible strap. Each end of the double-ended flexible strap may be inserted into opposing interarch receptacles or ratchet heads to fix the patient's jaws together, as is described in more detail in relation to the figures. The various embodiments of the flexible straps are inserted through the port on the ratchet head, thereby engaging the ratchet teeth on the flexible strap with the pawl and allowing insertion of the flexible strap but not its removal from the port. In some embodiments the flexible straps are flexible along their length in one or both axes perpendicular to the longitudinal axis of the strap, so that they may be bent, curved or twisted in simple or compound curves as necessary. In some embodiments the flexible straps have limited extensibility or compressibility parallel to their longitudinal axis.

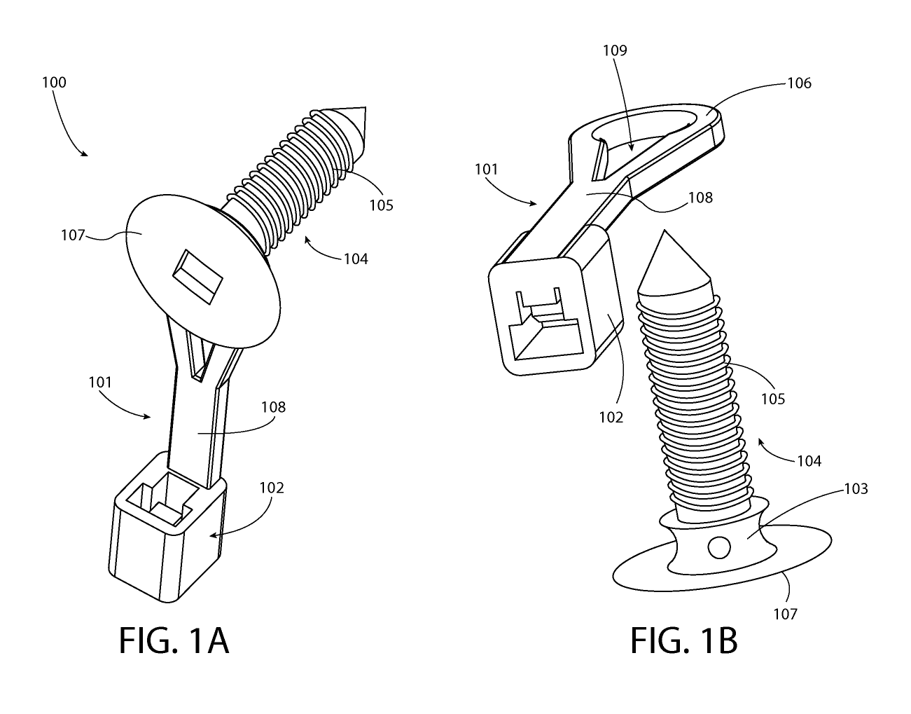

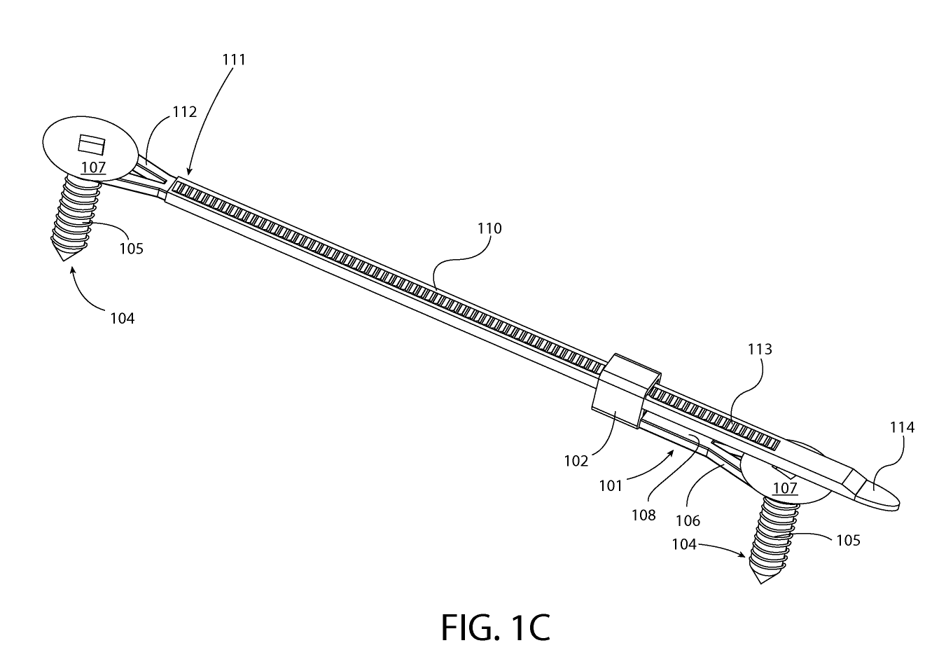

As best shown in FIGS. 1A and 1B, a first embodiment of an intermaxillary fixation accessory is a unique hanger assembly 100 that provides an interarch receptacle or ratchet head 102 for connecting to a bone screw 104 to be fastened into a patient's jaw bone. The screw 104 includes a threaded shaft 105 and a driving head 107 at one end of the threaded shaft 105. A receptacle hanger 101 comprises an interarch receptacle or ratchet head 102 and a washer 106 attached to ratchet head 102 by spacer bar 108. In some embodiments the washer 106 is elongated or slotted and is provided with a hole 109. When in use the shaft 105 of bone screw 104 is disposed through the hole 109 in washer 106 and washer 106 is disposed adjacent to head 107 of bone screw 104. The spacer bar 108 separates the ratchet head 102 a predetermined distance from the washer 106. The port of the ratchet head 102 is disposed substantially perpendicular to the washer so that a flexible strap secured in the ratchet head extends substantially perpendicular to a bone screw inserted through the hole in the washer.

When installed in a patient's mouth, the bone screw 104 is fastened into the jaw bone of the patient in a location where one or more teeth are missing. The installed hanger assembly 100 disposes the ratchet head 102 adjacent to the areas of missing teeth so that an interarch receptacle on an arch bar may be secured to the ratchet head 102 and thus to the jaw. In some embodiments, bone screw 104 may be provided with a bearing area 103 disposed on the shaft 105 of the bone screw 104 adjacent to the head 107. The bearing area provides a surface for contact with the washer 106 to allow the washer to rotate smoothly. The surface of bearing area 103 may be flat, concave, convex or textured as desired.

As shown in the previously referenced Shah patent, interarch receptacles are positioned along the arch bar for the receipt and retention of a flexible strap or the male end of a zip tie. In use, the inventive receptacle hanger 101 is positioned on the patient's jaw opposite an interarch receptacle, or zip tie receptacle, mounted on the arch bar or ratchet head attached to another bone screw 104 on the opposing jaw of the patient.

For example, if the patient presented with no teeth on the left upper jaw but has teeth on the lower left jaw, an arch bar may be fastened to the lower teeth. A bone screw 104 could be mounted in to the upper left jaw bone of the patient opposite the interarch receptacles presented on the arch bar fastened to the left lower teeth. In some embodiments the washer 106 of the receptacle hanger 101 may be placed over the threaded shaft 105 on the screw 104 and simultaneously mounted with the bone screw 104. In other embodiments the slotted or wide portion of the washer 106 may be slipped over the driving head 107 of the bone screw 104 after it has been secured in the patient's jaw bone. The interarch receptacle 102 is generally oriented so that a zip tie or flexible strap having reversed teeth at opposite ends can be inserted in to both the interarch receptacle and the opposed ratchet head 102 so that when the patient's jaws are closed together the zip tie or flexible strap holds the opposing arch bar receptacle and ratchet head 102 and associated arch bar (on the lower teeth) in place.

In situations where multiple teeth are missing from both the upper and lower jaw of the patient, it may be necessary to use multiple bone screws 104 and receptacle hangers 101 on both the upper and lower jaw to present enough ratchet heads so that the jaws are maintained in the preferred rigid closed position by the flexible straps.

Referring now to FIG. 1C, in some embodiments of the invention a flexible strap 110 with integral washer 112 is utilized. The innovative washer 112 is similar in shape and configuration to embodiments of washer 106, and is attached to or integrally formed as part of a first end 111 of flexible strap 110. The flexible strap 110 is provided with ratchet serrations 113 on at least one surface thereof for engaging the ratchet head 102. When the bone screws 104 are disposed in opposing jaw bones of a patient, washers 112 and 106 may be engaged on the bone screws 104 as described in relation to FIG. 1. The second end 114 of flexible strap 110 is inserted in the opposite ratchet head 102 and pulled until the jaws are in a closed position to hold the patient's jaws together. A physician may alternate using a bone screw with either a washer 112 or a receptacle hanger 101 as appropriate in the circumstances.

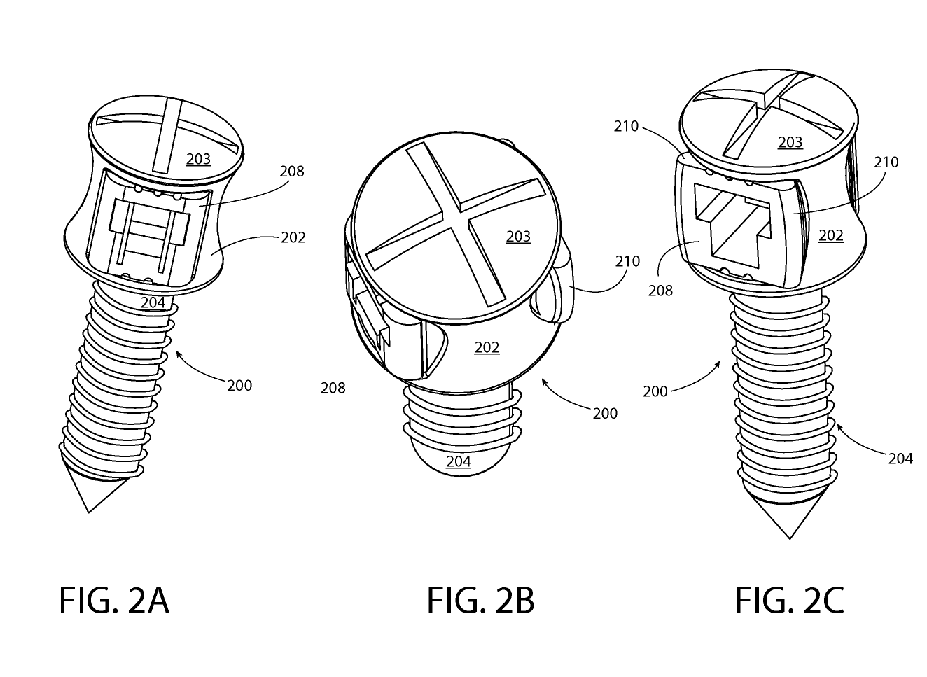

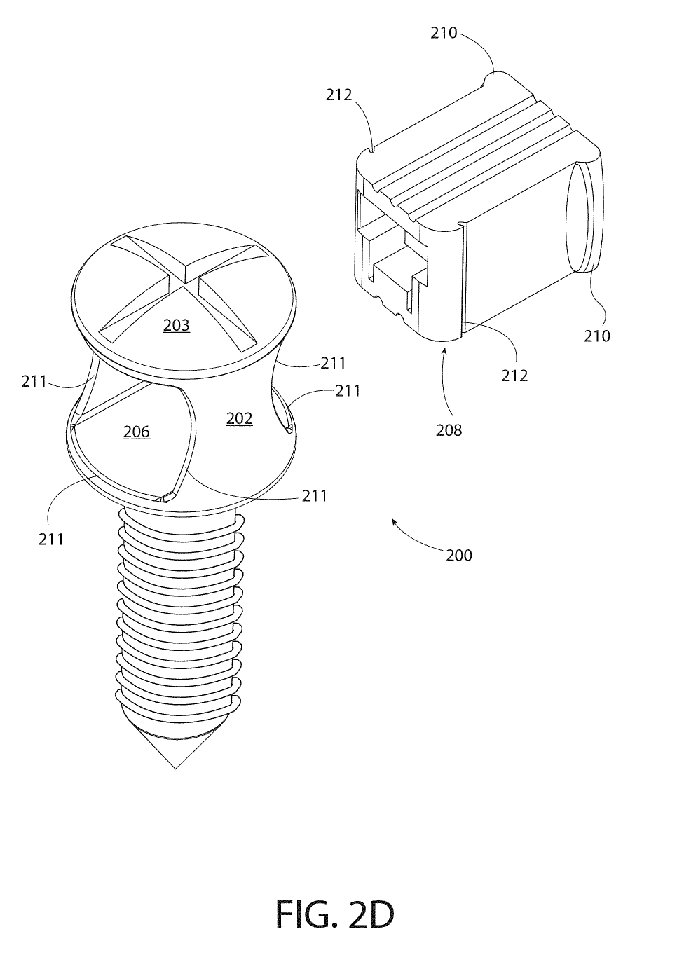

Referring now to FIGS. 2A-2D, in another embodiment of an intermaxillary fixation accessory, a zip tie compatible or flexible strap compatible bone screw 200 is utilized for intermaxillary fixation. The bone screw 200 includes an elongated driving head 202 for engagement with a driving tool and a threaded shaft 204. The elongated driving head 202 extends upwardly from the shaft 204 of the bone screw, and the depicted embodiment of the head 202 is generally cylindrical but may also have a hexagonal or other shaped cross-section. In the depicted embodiment the top surface 203 of the driving head 202 is provided with structures for engaging a driving tool such as a square or phillips screw driver, while in other embodiments the shape of driving head 202 may be capable of engagement by a tool such as a wrench. Within the elongated driving head 202 a ratchet head is provided for receiving a flexible strap. The ratchet head may be integrally formed as part of the elongated driving head 202, or it may be removable as depicted in FIGS. 2A-2D.

In some embodiments, the driving head 202 includes a cavity 206 for receiving a removable ratchet head 208. The cavity 206 is an opening laterally through the elongated driving head 202 of the bone screw 200 in to which a ratchet head 208 may be inserted. The ratchet head 208 may be frictionally retained, adhered or otherwise secured in cavity 206. The cavity 206 may include a small ledge or other retention member along one edge 211 of the cavity 206 to further engage and retain the ratchet head 208 in place. In some embodiments, the ratchet head 208 may be formed with at least one lip or protrusion 210 that engages the driving head 202 adjacent the cavity 206 to prevent the ratchet head 208 from passing through the cavity 206. In some embodiments, ratchet head 208 may also be provided with slots or flanges 212 to engage the edge 211 of cavity 206 to engage and retain the ratchet head 208 in cavity 206.

The bone screw 200 is installed by driving the threaded shaft 204 into the bone of the patient. The openings of the cavity 206 are preferably oriented in a vertical position with respect to the jawline of the patient. When the screw 200 is installed in the jaw of a patient, the ratchet head 208 is disposed in cavity 206 with protrusion 210 disposed generally on the side of driving head 202 away from the opposite jaw. A flexible strap such as 110 is then installed into the ratchet head 208 by passing one end of the flexible strap through the ratchet head 208. When the other end of the flexible strap is attached to another interarch receptacle, ratchet head, or bone screw and tightened to hold the jaws of the patient together, the flexible strap will pull interarch receptacle 208 into cavity 206 maintaining protrusion 210 securely against the edge of cavity 206. The protrusion 210 engages a portion of the driving head 202 adjacent to the cavity 206 such as edges 211 and prevents the ratchet head 208 from passing through the cavity 206. Where opposing screws are presented in both the upper and lower jaw, a reversing zip tie or double-ended flexible strap, where the teeth on one end of the flexible strap are reversed from the teeth on the other end, is provided. The teeth disposed in opposite directions allow each end of the double-ended flexible strap to be inserted into opposing interarch receptacles or ratchet heads for securing the opposite jaws together. This double-ended flexible strap is positioned within and manipulated through the ratchet head 208 and secured by the ratchet inside the ratchet head 208 engaging the teeth on the flexible strap. As the reversing zip tie or double-ended flexible strap is pulled through the ratchet head 208 and through the opposed interarch receptacle or ratchet head, the patient's jaws are forced together and secured in a closed position.

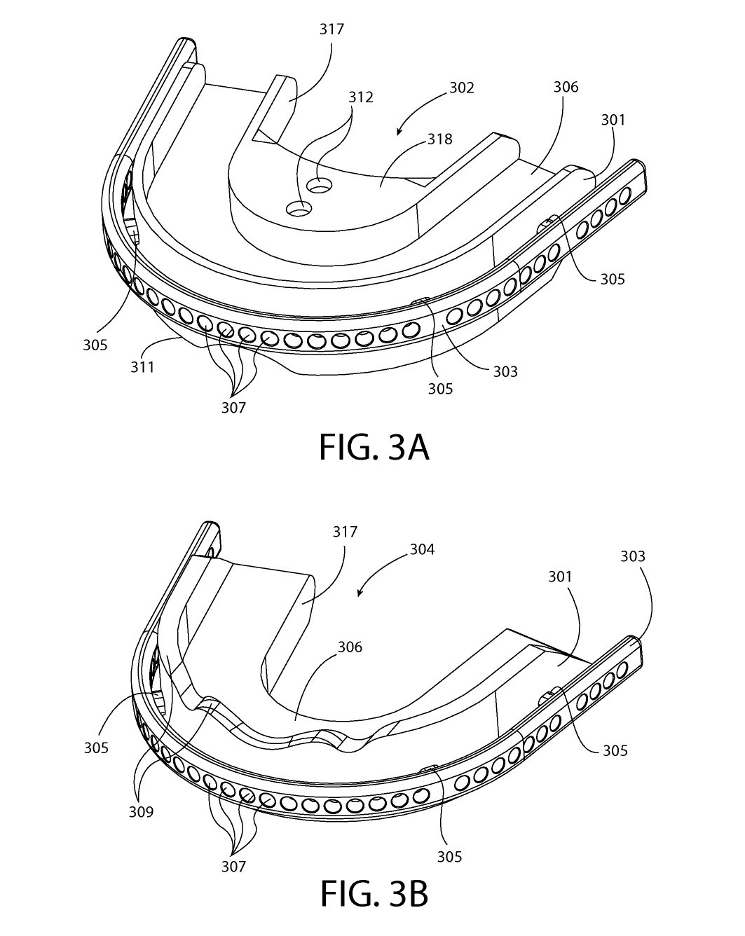

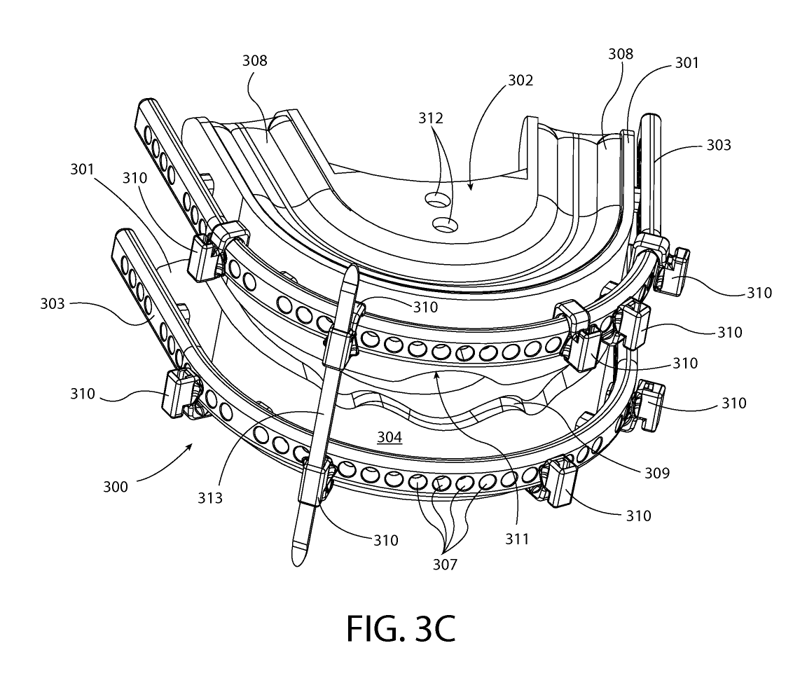

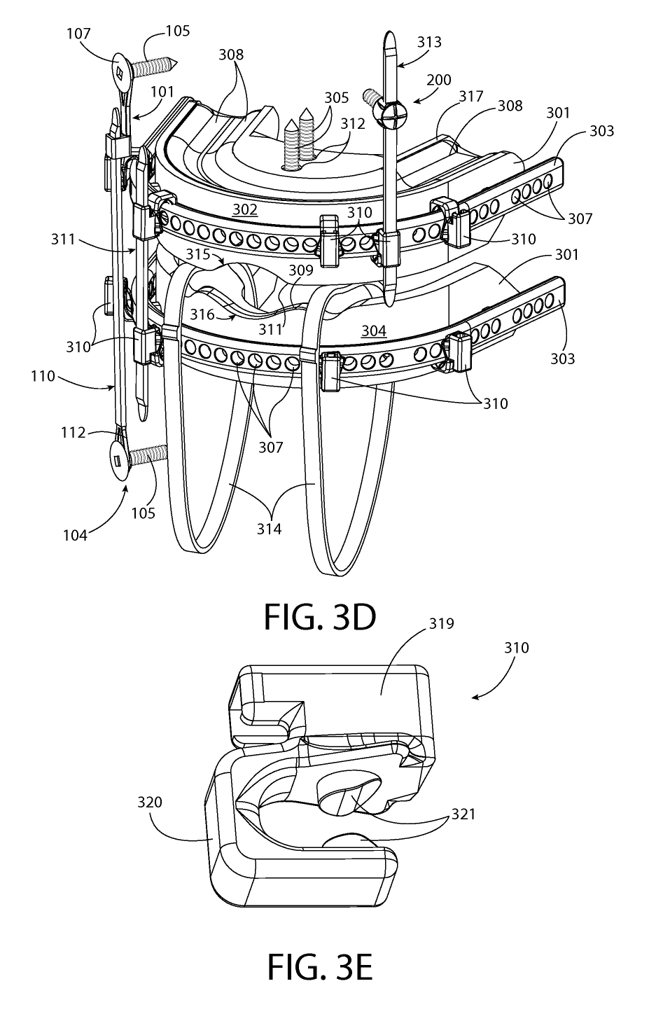

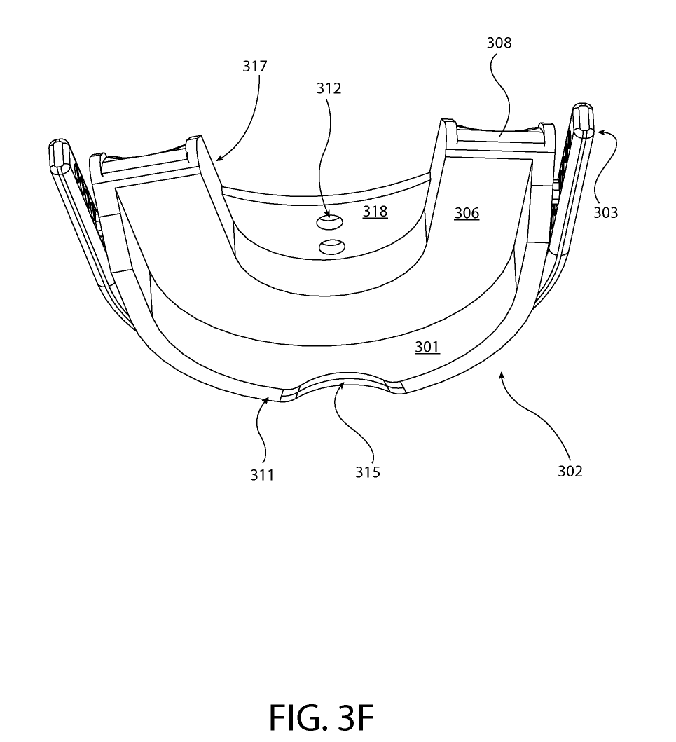

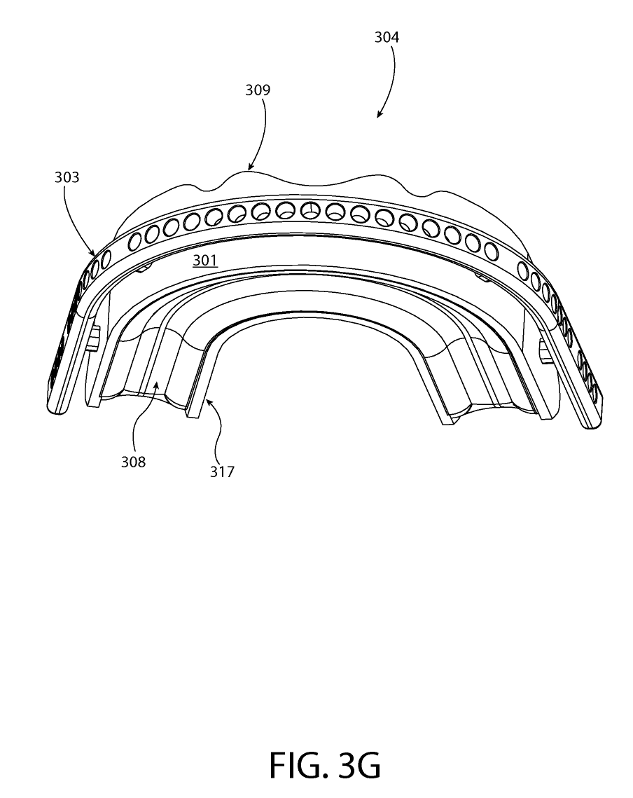

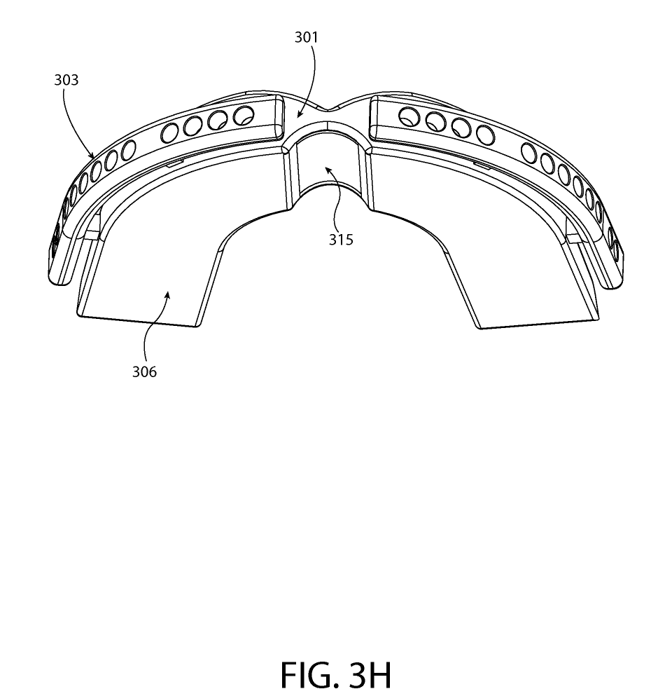

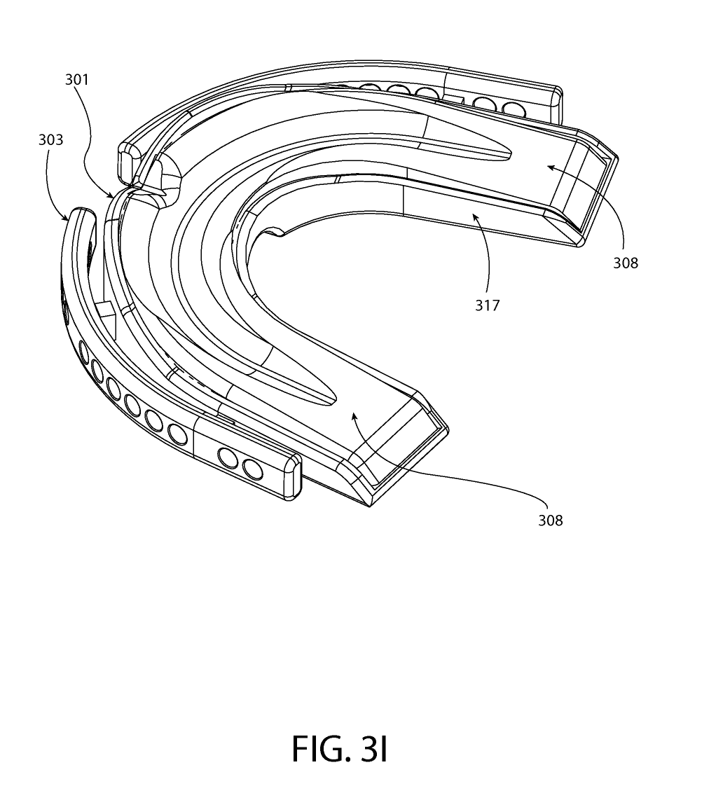

In those instances when a patient presents with no teeth whatsoever on one or both jaws, and dentures are not provided or are otherwise not available, a different problem must be overcome. It is not desirable for bone healing to fix the patient's upper and lower jaws together when there are no teeth or dentures present. Doing so results in improper jaw bone alignment during the healing process and may make subsequent use of dental implants or dentures difficult, if not impossible. To overcome this problem, an inventive set of dental blocks (sometimes referred to as dental splints) 300 has been designed and developed, an embodiment of which is depicted in FIGS. 3A-3D.

An upper dental block 302, shown in FIGS. 3A and 3F, is suitable for replacement of the upper teeth and a lower dental block 304, shown in FIGS. 3B and 3G, is designed for replacement of the lower teeth. The blocks 302 and 304 may be comprised of substantially rigid portion or plate 306 designed to take the place of the patient's teeth and a soft, formable, malleable, or flexible or compressible portion 308 (shown in FIGS. 3C and 3D) that engages the patient's gum when the blocks 302 and 304 are in place. The general shape of the blocks 302 and 304 are semi-arcuate when viewed from above. In some embodiments they comprise a substantially semi-circular front portion with substantially linear wings extending substantially tangentially to the circumference of the semi-circular portion. In some embodiments the plate 306 is substantially flat and extends perpendicularly to the outside plate 301. In some embodiments an inside plate 317 is provided adjacent to the inside edge of plate 306 and substantially perpendicular to the plate 306. The inside plate 317 and outside plate 301 generally run around the inner and outer periphery, respectively, of the patient's gums.

Blocks 302 and 304 may be provided in various sizes to fit different patient's bites. When in place, the dental blocks 302 and 304 hold the upper and lower jaws apart a sufficient distance such that when the jaw bones heal, suitable space has been provided between the jaw bones for proper placement of dental implants or dentures. If a patient has teeth on one jaw but not on the other, only one of blocks 302 and 304 may be necessary.

In some embodiments, each dental block 302 and 304 is provided with multiple receptacles or ratchet heads for receiving the male end of a zip tie or a flexible strap. In some embodiments, the ratchet heads or receptacles may be fixedly or pivotally attached directly to the blocks 302 and 304 at various locations around the outside plate 301 of each block 302 and 304. In the embodiment shown in FIGS. 3A-3D, an attachment rail 303 is provided on each dental block 302 and 304. The rail 303 extends around the outside plate 301 of the dental blocks 302 and 304. In the depicted embodiment, the rail 303 is supported slightly separated from the outside plate 301 by a plurality of posts 305. In other embodiments the rail 303 may be continuously connected to outside plate 301 or may be formed on or attached to discrete segments of outside plate 301. Rail 303 has a generally rectangular cross-section, but may be provided with beveled or rounded edges as depicted in the figures, or may be circular in cross-section.

Rail 303 provides a means of attaching a plurality of ratchet heads or receptacles 310 to the rail for attaching the dental blocks 302 and 304 to a patient's jaws and to the other dental block. In the depicted embodiment, bores, holes or indentations 307 are provided at numerous locations along the length of rail 303. If indentations that do not extend completely through rail 303 are provided at locations 307, a corresponding indentation may be provided on the inside surface of rail 303 facing the outside plate 301. In some embodiments holes 307 will extend completely through rail 303. In some embodiments, receptacles 310 may be provided with screws or locking pins for inserting into holes 307. In other embodiments, such as that shown in FIGS. 3C and 3D, receptacles or ratchet heads 310 may be provided with clip elements that clip over the rail 303 and engage the holes 307 from one or both sides with protrusions on the clip. The rail 303 with a plurality of receptacle mounting locations 307 allows for the positioning of the receptacles to be configured based on the circumstances and condition of the patient's teeth, gums and jaws.

The upper edge 309 of the outside plate 301 of lower dental block 304 may be provided with various undulations, lower portions, higher portions, raised portions, or indentations along the length of the outside plate 301. Similar undulations, raised portions or indentations may be provided on lower edge 311 of upper dental block 302. The varying shape of the upper edge 309 and lower edge 311 may be designed to interact with the patient's teeth and the other dental block to create and maintain space for the patient's tongue and passageways between the dental blocks for airways, tubes, circum-mandibular straps, and other medical devices when the two blocks are in contact at the outside plates.

Referring now to FIG. 3C, an embodiment of the upper dental block 302 and the lower dental block 304 are depicted. A plurality of clip on receptacles or ratchet heads 310 have been attached to the rail 303 and a double-ended flexible strap 313 inserted into two opposing receptacles to prevent separation of the dental blocks 302 and 304. Other double-ended flexible straps 313 would also be inserted at other locations with receptacles 310. As can be seen in this figure, the clip on receptacles 310 are clipped on to the rail with the clip disposed away from the direction of force on the flexible strap that will be inserted into the receptacle. This orients the ratchet within the receptacle 310 in the necessary direction to engage the teeth on the flexible strap.

The dental blocks 302 and 304 are held in place using methods described herein for securing dentures in place, some of which are depicted in FIG. 3D. The upper dental block may include screw holes 312 for fastening the block in place with screws placed in the patient's palate. The holes 312 may be in an extension of plate 306 or an additional plate 318 attached to the inside wall 317 of the upper dental block 302.

Unique circum-mandibular straps 314 may be used to secure the lower dental block 304 in place. The circum-mandibular straps 314 are placed by methods similar to known methods of wiring lower dentures in place for intermaxillary fixation procedures. For securing the lower dental block 304 with circum-mandibular straps 314, as best shown in FIG. 3D, a hole is generally made between the patient's gum and cheek tissue oriented downward and passing along the patient's jaw, exiting under their chin. A second hole is then bored upwardly through the floor of the patient's mouth adjacent the inner periphery of the gum. This bore is generally made with a pointed or sharp instrument such as an awl or trochar. The lower dental block 304 is then placed over the patient's lower gums so that the gums engage and support the soft insert 308. Although not shown in FIG. 3C, a soft insert 308 is also provided on the lower surface of dental block 304 for contacting the lower gums of the patient. The circum-mandibular straps 314 are inserted through the hole between the patient's cheek and gum downward below the patient's chin and then back up through the bore that is interior the patient's gums. The circum-mandibular straps 314 are passed over the dental block 304 so that the dental block is pulled downward on to the patient's gum and retained rigidly in place. The male end of the circum-mandibular strap is then inserted into the ratchet head disposed on the other end thereof, and secured by the pawl and ratchet teeth on the strap. This procedure generally requires at least two circum-mandibular straps 314, one on either side of the patient's mouth. As previously described, the dental blocks may be provided with grooves on edges 309 and 311 into which the circum-mandibular straps 314 are seated to prevent unwanted shifting, sliding or other movement of the circum-mandibular straps 314 along the dental blocks once they have been placed.

Two perspective views of a similar embodiment of the dental block 302 is depicted in FIGS. 3H and 3I. Similar numbers identify similar components of the depicted dental block. The embodiment depicted in FIGS. 3H and 3I may be used on either jaw and two of the depicted dental blocks may be used simultaneously on both jaws.

Referring now to FIG. 3D, in some embodiments a ratchet head or receptacle 310 is attached to one of the dental blocks 302 and 304 and may be secured to the jaw by a double-ended flexible strap 313 connected to a bone screw 104 and receptacle hanger 101 or a flexible strap compatible bone screw 200, with the bone screw 104 or 200 fixed in the patient's jaw. In some embodiments, the patient's jaws may be secured to one another using a bone screw 104 and receptacle hanger 101 fixed in one jaw, and bone screw 104 fixed in the other jaw, with a flexible strap 110 with integral washer 112 secured on the bone screw 104 and inserted into the receptacle hanger 101 attached to the opposing jaw.

Once the dental blocks are fixed in place, reverse zip ties or double-ended flexible straps 313 can be used in the receptacles 310 on the opposing dental blocks or opposing teeth to fasten the upper and lower jaws together. The edge 311 of upper block 302 may include an upper arcuate opening 315 and the lower block 304 may include a substantially similar, but opposing lower arcuate opening 316 so that when the dental blocks are in place, an annular opening is formed for access to the patient's mouth for cleaning, suction and the like, or in some instances, the placement of a breathing tube.

Referring now to FIG. 3E, an embodiment of a ratchet head 310 for use with a rail 303 is depicted. The ratchet head includes ratchet body 319 which contains the opening and pawl for receiving and retaining a flexible strap. A clip portion or member 320 extends from the side of the ratchet body 319 and fits around rail 303 so that it may be clipped on to the rail 303 at the desired location. The clip portion 320 contains two protrusions 321 that engage the holes 307 on rail 303 to secure the ratchet head 310 in place on the rail 303. The protrusions 321 may be sloped (as shown in the figures) or otherwise shaped to allow them to be clipped onto the rail but to resist removal of the clip portion 320 from the rail 303.

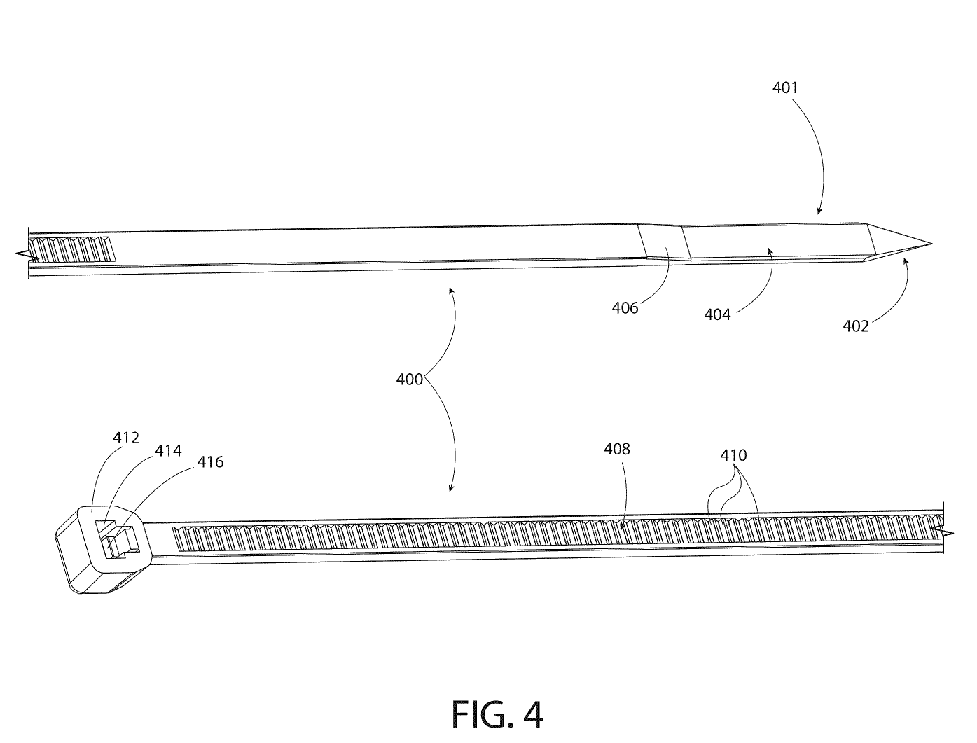

One of the inventive intermaxillary fixation accessories that can be used to place circum-mandibular straps 314 is an improved zip tie assembly or strap, one embodiment of which is shown in FIG. 4. The zip tie or circum-mandibular strap 400 has a dissection tip 402 at the terminus of a first or male end 401 of the strap 400. This dissection tip 402 may be conical or blade-shaped and sharp enough to allow the first end of the strap 400 to be pushed through the tissues surrounding a patient's jaws. In some embodiments the tip 402 is preferably integral to the strap 400 and formed from plastic that can be provided with a sharp edge, if needed, to facilitate advancement of tip 402 through tissue. In some embodiments the tip 402 may be separate from and attached to the male end of strap 400. In some embodiments, the dissection tip 402 may be made of metal and molded onto the plastic of the zip tie. A sharp blade may take the place of the dissection tip 402 for some applications.

In some embodiments, the male end 401 of strap 400 may have one or more sections 404 with a thinner profile or smaller cross-section than other portions of the strap 400 to ease insertion of the strap 400 through the patient's tissue. Between the sections 404 of the strap 400, some embodiments incorporate sloping sections 406 gradually narrowing the thickness or cross-section of strap 400 as it approaches the dissection tip 402.

Strap 400 is provided on one surface with a gear rack section 408 provided with a plurality of ratchet teeth 410. The second end of strap 400 is provided with ratchet head 412 with a port 414 through the head 412. Inside the port 414 a pawl 416 is provided to engage ratchet teeth 410. In some embodiments, the other ratchet heads described herein contain similar elements. As with the other straps used in the depicted embodiments of the invention, when the first end 401 of strap 400 is inserted through the port 414 in the ratchet head 412, the ratchet teeth 410 engage pawl 416 to allow the strap to be pulled through port 414 but not to be retracted from the port 414.

In practice, the dissection tip 402 of the strap 400 is used like an awl or trochar for wire placement. The strap 400 may be pushed through the tissue adjacent the gum and then passed below the patient's chin, creating a small loop and then back up through the tissue so the strap 400 encircles the jaw and any associated dentures or dental block. As the strap 400 is pushed through the tissue, dissection tip 402 cuts the tissue sufficiently to allow the strap 400 through the tissue.

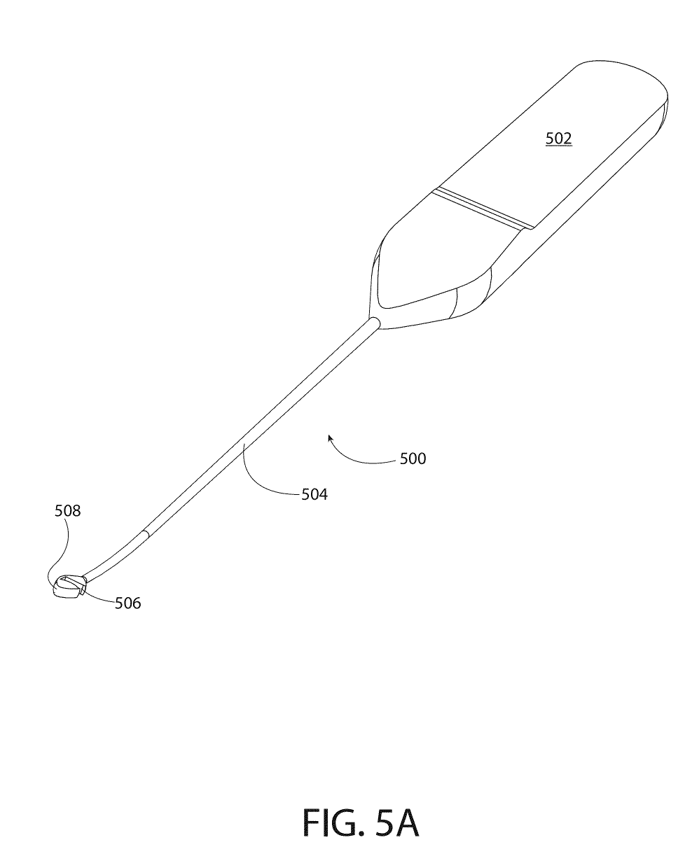

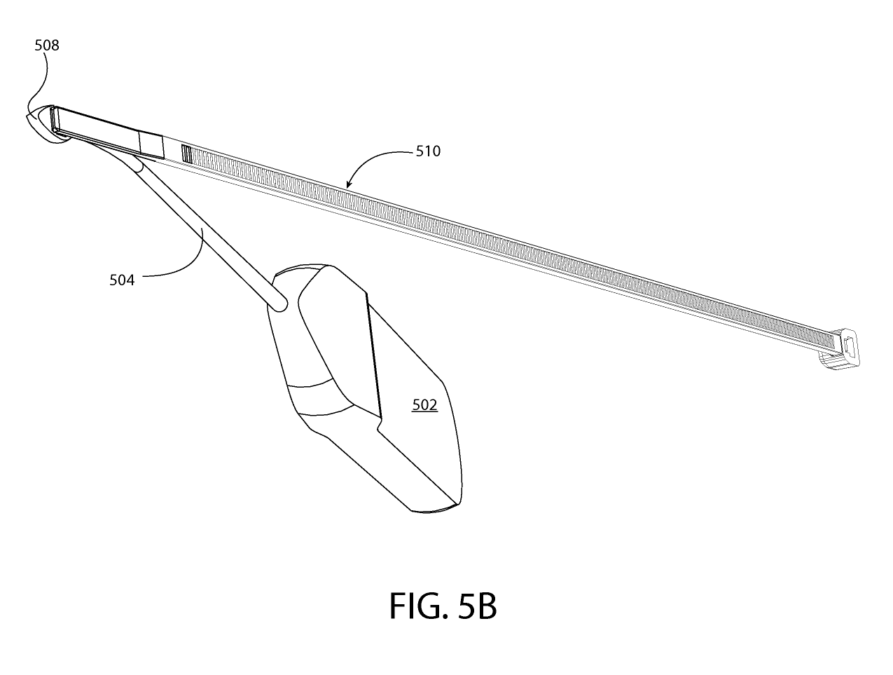

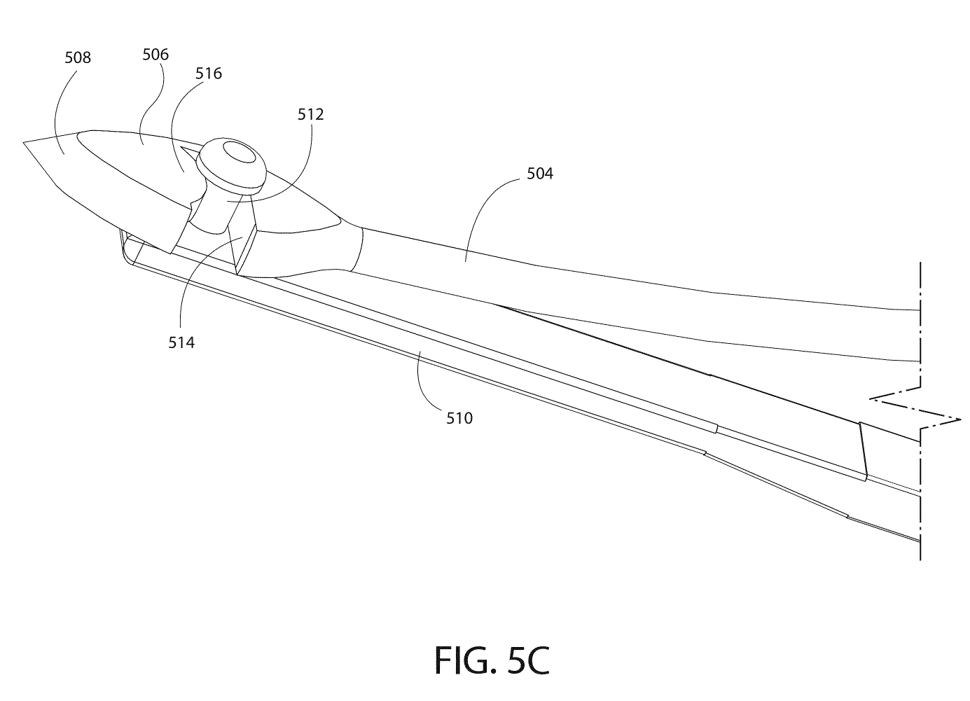

Another inventive tool, as shown in FIGS. 5A, 5B, and 5C, is a unique tool 500 that is provided with a handle 502, a shaft 504 and a unique retention member 506 at or substantially near the blade or dissection tip 508 of the tool 500. In one embodiment, the retention member 506 is configured to engage and retain a knob or pin 512 provided near the first end of an embodiment of circum-mandibular strap 510. In the depicted embodiment, the retention member 506 is provided with a retention slot 514 formed at or near the blade 508. The blade 508 may be wider than the flexible strap in some embodiments. Further, the retention member 506 may include a nodule or detent 516 within the slot 514 to further engage and retain the knob 512 of the circum-mandibular strap 510. The tool 500 is used in a manner similar to a trochar or awl to form holes in the patient's jaw or adjacent tissues for placing straps as described above to secure dentures or a dental block. As the blade end 508 advances through tissue, it pulls the first end of the circum-mandibular strap 510 through the tissue so that it is placed simultaneously as the tool 500 is advanced. Once the circum-mandibular strap 510 has been placed through the patient's jaw as described above, the knob 512 is disengaged from the slot 514 on the retention member 506. Tool 500 may then be retracted back through the hole in the patient's jaw, leaving the strap 510 in place. The knob 512 or the end of strap 510 where knob 512 is attached, may be cut off the strap 510 so that the remainder of the strap 510 may be inserted into and secured by ratchet head 412.

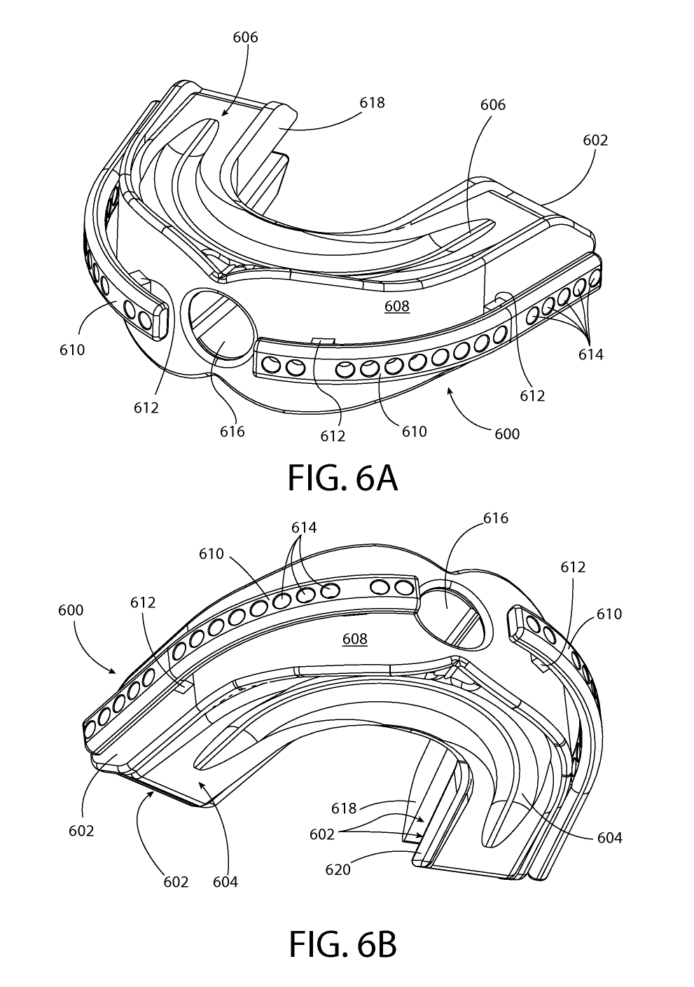

Referring now to FIGS. 6A and 6B, an additional embodiment of a dental block is depicted. This embodiment is a single piece dental block that may be used with patients that have no teeth on either upper or lower jaws. The dental block 600 is provided with a rigid plate 602 shaped in the approximate shape of the human jaw for receiving the gums of a patient. Cushions 604 and 606 are provided on the top and bottom of the block 602 to cushion the gums of the patient. An outer plate 608 is attached to the rigid block 602. A rail 610 is attached to the outer surface of plate 608. In some embodiments, the rail 610 may be integrally formed with the plate 608 or may be separated from plate 608 by a gap and supported by posts 612. Rail 610 is provided with holes or indentations 614 similar to holes 307 described in relation to an earlier figure. In some embodiments, the plate 602 may comprise two slightly separated upper and lower plates attached to the outside plate 608. In some embodiments, plates 602 may have inner plates 618 and 620 attached to the inner edge thereof.

The general shape of the block 600 is semi-arcuate when viewed from above. In some embodiments the block comprises a substantially semi-circular front portion with substantially linear wings extending substantially tangentially to the circumference of the semi-circular portion. In some embodiments the plate 602 is substantially flat and extends perpendicularly to the outside plate 608. In some embodiments the inside plates 618 and 620 are provided adjacent to the inside edge of plate 602 and substantially perpendicular to the plate 602. The inside plates 618 and 620 and outside plate 608 generally run around the inner and outer periphery, respectively, of the patient's gums.

In some embodiments, a port 616 may be provided through the dental block 602. The port 616 may be used to suction the patient's mouth, insert tubes for air, nutrition or other needs, or other similar purposes.

As shown in FIGS. 3C and 3D, ratchet heads 310 may be attached to rail 610 and used to attach the dental block to the upper and lower jaws of a patient using the various bone screws and other accessories described herein.

Many different arrangements of the various components depicted, as well as components not shown, are possible without departing from the spirit and scope of the present invention. Embodiments of the present invention have been described with the intent to be illustrative rather than restrictive. Alternative embodiments will become apparent to those skilled in the art that do not depart from its scope. A skilled artisan may develop alternative means of implementing the aforementioned improvements without departing from the scope of the present invention.

It will be understood that certain features and subcombinations are of utility and may be employed without reference to other features and subcombinations and are contemplated within the scope of the claims. Not all steps listed in the various figures need be carried out in the specific order described.

What is claimed is:

1. A system for intermaxillary fixation of a patient's jaw bone using at least one bone screw and at least one flexible strap having ratchet teeth, the system comprising:
at least one intermaxillary fixation accessory capable of attachment to a patient's jaw using a bone screw;
wherein the at least one intermaxillary fixation accessory incorporates a ratchet head for receiving a flexible strap;
wherein the ratchet head comprises a case having a port for receiving a flexible strap, and a pawl disposed in the port of the ratchet head to engage ratchet teeth on a flexible strap to allow insertion of a flexible strap into the ratchet head but to prevent removal of a flexible strap from the ratchet head.

2. The system for intermaxillary fixation of claim 1 wherein the intermaxillary fixation accessory further comprises:
a washer for mounting onto a bone screw;
wherein the ratchet head is disposed to the side of the washer and is fixedly attached to the washer, and
wherein the port of the ratchet head is disposed substantially perpendicular to the washer.

3. The system for intermaxillary fixation of claim 2 wherein the washer is provided with an elongated hole having a wide portion and a narrow portion, wherein the wide portion is capable of fitting over the driving head of a bone screw, and the narrow portion is not capable of fitting over the driving head of a bone screw.

4. The system for intermaxillary fixation of claim 1 wherein the intermaxillary fixation accessory further comprises:
a bone screw having a shaft for implantation in a jaw bone and an elongated driving head;
wherein the ratchet head is incorporated into the elongated head of the bone screw.

5. The system for intermaxillary fixation of claim 3 wherein the ratchet head is removably incorporated into the elongated head of the bone screw, and a cavity is provided in the elongated driving head for receiving the removable ratchet head.

6. The system for intermaxillary fixation of claim 2 or 4 further comprising:
at least one dental block for maintaining a desired space between a patient's jaw bones, the dental block comprising a rigid plate for receiving a patient's gums, the rigid plate having a semi-arcuate shape, and an outer plate attached to an outer edge of the rigid plate and substantially perpendicular to the rigid plate;
at least one ratchet head attached to the at least one dental block for receiving a flexible strap.

7. The system for intermaxillary fixation of claim 6 wherein:
the at least one dental block further comprises a rail attached to the outer plate of the dental block; and
the at least one ratchet head is removably attached to the rail.

8. The system for intermaxillary fixation of claim 7 wherein:
the rail is provided with a plurality of holes through or indentations in the rail for removably attaching the at least one ratchet head; and
each of the at least ratchet head further comprises a clip member having protrusions for engaging one of the plurality of holes or indentations on the rail.

9. The system for intermaxillary fixation of claim 8 wherein the at least one dental block further comprises a malleable insert attached to the rigid plate, the insert to contact a patient's gums.

10. The system of intermaxillary fixation of claim 9 wherein a hole is provided through the outside plate of the at least one dental block for passage of tubes, air and other items through the dental block.

11. The system of intermaxillary fixation of claim 9 wherein the at least one dental block comprises an upper dental block and a lower dental block for attachment to the upper and lower jaw of a patent, respectively, and a ratchet head attached to the upper dental block is secured to a ratchet head attached to the lower dental block to secure the upper dental block to the lower dental block.

12. The system of intermaxillary fixation of claim 11 wherein a ratchet head attached to the upper dental block is attached to at least one intermaxillary fixation accessory for attachment to a patient's upper jaw, and wherein a ratchet head attached to the lower dental block is attached to at least one intermaxillary fixation accessory for attachment to a patient's lower jaw.

13. The system of intermaxillary fixation of claim 11 wherein the upper dental block is secured to a patient's palate by at least one bone screw secured through at least one hole in the rigid plate of the upper dental block.

14. The system of intermaxillary fixation of claim 11 wherein the top and bottom edges of the outside plates attached to the lower and upper dental blocks, respectively, are provided with lower and higher portions that create passageways between the upper and lower dental blocks when the dental blocks are secured together.

15. The system of intermaxillary fixation of claim 10 wherein the at least one dental block comprises a single dental block having an upper insert and a lower insert for contacting a patient's upper and lower gums, respectively, and wherein at least one ratchet head attached to the dental block is attached to at least one intermaxillary fixation accessory for attachment to a patient's upper jaw, and at least one ratchet head attached to the dental block is attached to at least one intermaxillary fixation accessory for attachment to a patient's lower jaw.

16. The system of intermaxillary fixation of claim 8 further comprising at least one washer attached to one end of a flexible strap having ratchet teeth, wherein the washer may be mounted onto a bone screw fastened into a patients jaw and the flexible strap may be secured to a ratchet head.

17. The system of intermaxillary fixation of claim 11 further comprising at least one circum-mandibular strap for securing the lower dental block to a patient's lower jaw by encircling the jaw and the lower dental block, the circum-mandibular strap comprising a ratchet head attached to first end of a flexible strap having ratchet teeth.

18. The system of intermaxillary fixation of claim 17 wherein the circum-mandibular strap further comprises a dissection tip attached to a second end of the flexible strap, the dissection tip for inserting the flexible strap through a patient's tissues to place the circum-mandibular strap around a patient's lower jaw.

19. The system of intermaxillary fixation of claim 17 further comprising:
a pin attached to and extending from the circum-mandibular strap near a second end thereof;
an awl having a handle, a shaft attached at a first end thereof to the handle, a blade attached to the second end of the shaft for cutting through a patient's tissues, and a retention slot in the blade for receiving the pin on the circum-mandibular strap;
wherein the retention slot receives the pin for pulling the circum-mandibular strap through a patient's tissues to place the circum-mandibular strap around a patient's lower jaw.

20. A system for intermaxillary fixation accessory comprising:
at least one dental block for maintaining a desired space between a patient's jaw bones, the dental block comprising a rigid plate for receiving a patient's gums, the rigid plate having a semi-arcuate shape, and an outer plate attached to an outer edge of the rigid plate and substantially perpendicular to the rigid plate;
at least one ratchet head attached to the at least one dental block for receiving a flexible strap.

21. The system for intermaxillary fixation of claim 20 wherein:
the at least one dental block further comprises a rail attached to the outer plate of the dental block; and
the at least one ratchet head is removably attached to the rail.

22. The system for intermaxillary fixation of claim 21 wherein:
the rail is provided with a plurality of holes through or indentations in the rail for removably attaching the at least one ratchet head; and
each of the at least ratchet head further comprises a clip member having protrusions for engaging one of the plurality of holes or indentations on the rail.

23. The system for intermaxillary fixation of claim 22 wherein the at least one dental block further comprises a malleable insert attached to the rigid plate, the insert to contact a patient's gums.

24. The system of intermaxillary fixation of claim 23 wherein a hole is provided through the outside plate of the at least one dental block for passage of tubes, air and other items through the dental block.

25. The system of intermaxillary fixation of claim 23 wherein the at least one dental block comprises an upper dental block and a lower dental block for attachment to the upper and lower jaw of a patent, respectively, and a ratchet head attached to the upper dental block is secured to a ratchet head attached to the lower dental block to secure the upper dental block to the lower dental block.

26. The system for intermaxillary fixation of claim 25 further comprising:
at least one intermaxillary fixation accessory capable of attachment to a patient's jaw using a bone screw;
at least one flexible strap having ratchet teeth;
wherein the at least one intermaxillary fixation accessory incorporates a ratchet head for receiving a flexible strap;
wherein the ratchet head comprises a case having a port for receiving a flexible strap, and a pawl disposed in the port of the ratchet head to engage ratchet teeth on a flexible strap to allow insertion of a flexible strap into the ratchet head but to prevent removal of a flexible strap from the ratchet head.

27. The system for intermaxillary fixation of claim 26 wherein the intermaxillary fixation accessory further comprises:
a washer for mounting onto a bone screw;
wherein the ratchet head is disposed to the side of the washer and is fixedly attached to the washer, and
wherein the port of the ratchet head is disposed substantially perpendicular to the washer.

28. The system for intermaxillary fixation of claim 27 wherein the washer is provided with an elongated hole having a wide portion and a narrow portion, wherein the wide portion is capable of fitting over the driving head of a bone screw, and the narrow portion is not capable of fitting over the driving head of a bone screw.

29. The system for intermaxillary fixation of claim 26 wherein the intermaxillary fixation accessory further comprises:
a bone screw having a shaft for implantation in a jaw bone and an elongated driving head;
wherein the ratchet head is incorporated into the elongated head of the bone screw.

30. The system for intermaxillary fixation of claim 29 wherein the ratchet head is removably incorporated into the elongated head of the bone screw, and a cavity is provided in the elongated driving head for receiving the removable ratchet head.

31. The system of intermaxillary fixation of claim 26 wherein a ratchet head attached to the upper dental block is attached to at least one intermaxillary fixation accessory for attachment to a patient's upper jaw, and wherein a ratchet head attached to the lower dental block is attached to at least one intermaxillary fixation accessory for attachment to a patient's lower jaw.

32. The system of intermaxillary fixation of claim 26 wherein the upper dental block is secured to a patient's palate by at least one bone screw secured through at least one hole in the rigid plate of the upper dental block.

33. The system of intermaxillary fixation of claim 26 wherein the top and bottom edges of the outside plates attached to the lower and upper dental blocks, respectively, are provided with lower and higher portions that create passageways between the upper and lower dental blocks when the dental blocks are secured together.

34. The system of intermaxillary fixation of claim 24 wherein the at least one dental block comprises a single dental block having an upper insert and a lower insert for contacting a patient's upper and lower gums, respectively, and wherein at least one ratchet head attached to the dental block is attached to at least one intermaxillary fixation accessory for attachment to a patient's upper jaw, and at least one ratchet head attached to the dental block is attached to at least one intermaxillary fixation accessory for attachment to a patient's lower jaw.

35. The system of intermaxillary fixation of claim 22 further comprising at least one washer attached to one end of a flexible strap having ratchet teeth, wherein the washer may be mounted onto a bone screw fastened into a patients jaw and the flexible strap may be secured to a ratchet head.

36. The system of intermaxillary fixation of claim 26 further comprising at least one circum-mandibular strap for securing the lower dental block to a patient's lower jaw by encircling the jaw and the lower dental block, the circum-mandibular strap comprising a ratchet head attached to first end of a flexible strap having ratchet teeth.

37. The system of intermaxillary fixation of claim 36 wherein the circum-mandibular strap further comprises a dissection tip attached to a second end of the flexible strap, the dissection tip for inserting the flexible strap through a patient's tissues to place the circum-mandibular strap around a patient's lower jaw.

38. The system of intermaxillary fixation of claim 36 further comprising:
a pin attached to and extending from the circum-mandibular strap near a second end thereof;
an awl having a handle, a shaft attached at a first end thereof to the handle, a blade attached to the second end of the shaft for cutting through a patient's tissues, and a retention slot in the blade for receiving the pin on the circum-mandibular strap;
wherein the retention slot receives the pin for pulling the circum-mandibular strap through a patient's tissues to place the circum-mandibular strap around a patient's lower jaw.

39. A method of using the system of claim 1 to fixate the lower jaw of a patient to the upper jaw of the patient comprising the steps of:
placing at least one intermaxillary fixation accessory in either the lower or the upper jaw of the patient;
securing a first end of a flexible strap to the at least one intermaxillary fixation accessory either integrally or using a ratchet head attached to the intermaxillary fixation accessory; and
securing a second end of the flexible strap to the opposing jaw of the patient.

40. The method of claim 39 wherein the step of securing a second end of the flexible strap to the opposing jaw of the patient comprises the steps of:
attaching a washer to the second end of the flexible strap;
securing the washer to the opposing jaw of the patient using a bone screw.

41. The method of claim 39 wherein the step of securing a second end of the flexible strap to the opposing jaw of the patient comprises the steps of:
disposing a dental block against the jaw of the patient;
securing the second end of the flexible strap in a ratchet head attached to the dental block;
attaching the dental block to the opposing jaw of the patient.

42. The method of claim 41 wherein the step of attaching the dental block to the opposing jaw of the patient comprises:
securing a first end of a second flexible strap to a ratchet head attached to the dental block;
securing a second end of the second flexible strap to the opposing jaw of the patient.

43. The method of claim 42 further comprising the steps of:
placing a second intermaxillary fixation accessory in the opposing jaw of the patient;
securing the second end of the flexible strap to the second intermaxillary fixation accessory.

44. The method of claim 41 wherein the step of attaching the dental block to the opposing jaw of the patient comprises:
providing a second dental block;
attaching the second dental block to the opposing jaw of the patient;
securing a first end of a second flexible strap in a ratchet head attached to the dental block; and
securing a second end of the second flexible strap in a ratchet head attached to the second dental block.

45. The method of claim 41 wherein the step of attaching the dental block to the opposing jaw of the patient comprises the steps of:
inserting a first end of a circum-mandibular strap through the tissue around the patient's lower jaw;
placing a lower dental block against the lower jaw bone of the patient;
securing the first end of the circum-mandibular strap through a ratchet head attached to a second end of the circum-mandibular strap so that the circum-mandibular strap encircles the lower dental block and the lower jaw of the patient.

46. The method of claim 45 wherein the step of inserting a first end of the circum-mandibular strap through the tissue around the patient's lower jaw comprises:
providing a dissection tip at the first end of the circum-mandibular strap;
urging the dissection tip through the tissue around the patient's lower jaw.

47. The method of claim 45 wherein the step of inserting a first end of the circum-mandibular strap through the tissue around the patient's lower jaw comprises:
providing a pin attached to the circum-mandibular strap adjacent to the first end thereof;
engaging the pin in the retention slot of a blade of an awl;
urging the blade of the awl through the tissue around a patient's jaw;
disengaging the pin from the retention slot;
cutting off the portion of the circum-mandibular strap to which the pin is attached.

48. A method of using the system of claim 36 to fixate the lower jaw of a patient to the upper jaw of the patient comprising the steps of:
Placing at least one circum-mandibular strap around the lower jaw of a patient and a lower dental block;
Securing a second end of the circum-mandibular strap through a ratchet head attached to a first end of the circum-mandibular strap;

49. The method of claim 48 further comprising the step of securing the lower dental block to an upper dental block attached to the patient's upper jaw.

50. The method of claim 49 wherein the upper dental block is attached to the patient's upper jaw by a flexible strap attached to the upper dental block and to at least one intermaxillary fixation accessory.

51. The method of claim 49 wherein the upper dental block is attached to the patient's upper jaw by at least one bone screw secured through a hole in the upper dental block to the patient's palate.

52. A method of using the system of claim 32 to fixate the lower jaw of a patient to the upper jaw of the patient comprising the steps of:
Attaching an upper dental block to the patient's upper jaw using at least one bone screw disposed through a hole in the upper dental block;
placing at least one intermaxillary fixation accessory in the lower of the patient;
securing a first end of a flexible strap to the at least one intermaxillary fixation accessory either integrally or using a ratchet head attached to the intermaxillary fixation accessory; and
securing a second end of the flexible strap to the upper dental block.
